(19) United States
(12) Patent Application Publication
(10) Pub. No.: US 2011/0125226 A1
(43) Pub. Date: May 26, 2011

(54) COORDINATED DELIVERY OF SOLITON WAVES THROUGH COUPLED MEDICAL INSTRUMENTS

(76) Inventors: Larry Lytle, Rapid City, SD (US); Alf-Kare Eide Riisnaes, Rapid City, SD (US); Kip Lytle, Rapid City, SD (US); Shawn Gab, Rapid City, SD (US)

(21) Appl. No.: 12/626,634

(22) Filed: Nov. 26, 2009

(51) Int. Cl. A61N 5/06

(52) U.S. Cl. 607/88

(57) ABSTRACT

Disclosed are a method, an apparatus, and a system of a coordinated delivery of soliton waves through coupled medical instruments. In one embodiment, a method includes coupling a first medical instrument to another medical instrument. The method also includes generating a first soliton wave through the first medical instrument at a first wavelength and at a first frequency. In addition, the method includes generating a second soliton wave through another medical instrument at a second wavelength and at a second frequency. The method also includes coordinating a delivery of the first soliton wave and the second soliton wave on a biological medium through an algorithm that controls a start of a sequence of pulsation of the diodes from the different instruments affecting the delivery of laser and diode light of the first medical instrument and another medical instrument.

### FIELD OF TECHNOLOGY

This disclosure relates generally to the field of medical instruments and in particular, to coordinated delivery of soliton waves through coupled medical instruments.

### BACKGROUND

A patient may suffer from a medical condition (e.g., arthritis, diabetes, fractures) in a variety of locations on a body of the patient. The medical condition may impair a daily routine of the patient. A medical instrument (e.g., a hand-held medical instrument) may not be able to deliver a treatment to the patient in a coordinated form (e.g., simultaneously, synchronously). It may be difficult for a treatment provider to deliver care to the patient while manually operating multiple medical instruments simultaneously. Moreover, the treatment provider may not be able to control the multiple medical instruments simultaneously. Therefore, the patient may not get relief and the medical condition may deteriorate.

### SUMMARY

A method, apparatus, and a system of coordinated delivery of soliton waves through coupled medical instruments are disclosed. In one aspect, a method includes coupling a first medical instrument to another medical instrument (e.g., a second medical instrument). The method also includes generating a first soliton wave through the first medical instrument at a first wavelength and at a first frequency. In addition, the method includes generating a second soliton wave through another medical instrument at a second wavelength and at a second frequency. The method also includes coordinating a delivery of the first soliton wave and the second soliton wave on a biological medium through control of delivery of a soliton wave of laser and diode light of the first medical instrument and another medical instrument. In several embodiments, the medical instrument may be a laser therapy device.

In several embodiments the treatment or therapy administered by the medical instrument to treat a biological medium may be referred to as, but is not limited to, low-level laser therapy (LLLT), laser biostimulation, laser irradiation, laser therapy, low-power laser irradiation, or low-power laser therapy. In several embodiments, the medial instrument may provide laser therapy or laser treatment to the biological medium.

In addition, the method may include canceling a first nonlinear effect and a first dispersive effect in a first region between a first emitting region of the first medical instrument and the biological medium to create the first soliton wave. The first dispersive effect may be a first dispersion relationship between the first frequency and a first speed of the first soliton wave. The method may also include canceling a second nonlinear effect and a second dispersive effect in a second region between a second emitting region of the second medical instrument and the biological medium to create the second soliton wave. The second dispersive effect may be a second dispersion relationship between the second frequency and a second speed of the second soliton wave.

When the first medical instrument is a primary device, the first wave length may be between 400 nanometers and 1000 nanometers. In an embodiment, the first wave length may be between 645 nanometers and 811 nanometers, and the first frequency may be between 0.5 hertz and 200,000 hertz. In an embodiment, the first frequency may be between 1 hertz and 20,000 hertz, and the first region may be approximately 16.4 centimeters squared in area in spot size or 45.7 millimeters in diameter. In an embodiment, when the first medical instrument is a primary device, a treatment time required may be approximately 3 or more minutes and the device may shut off automatically after treatment or the device may run continuously and shut off under direction of the user. In addition, when the first medical instrument is a primary device, a treatment time required may be approximately 3 or more minutes on the biological medium, and an energy produced through the first medical instrument may be between 1 to 60 millijoules per second. In an embodiment, an energy produced through the first medical instrument may be approximately 21 millijoules per second, and a power generated may be less than 42 milliwatts.

Similarly, when the second medical instrument is a primary device, the second wavelength may be between 400 nanometers and 1000 nanometers. In an embodiment, the second wave length may be between 645 nanometers and 811 nanometers, and the second frequency may be between 0.5 hertz and 200,000 hertz. In an embodiment, the second frequency may be between 1 hertz and 20,000 hertz, and the second region may have an area of 16.4 centimeters squared in spot size. Also, when the second medical instrument is a primary device, a treatment time required may be approximately 3 or more minutes on the biological medium, an energy produced through the second medical instrument may be approximately 21 millijoules per second, and a power generated may be less than 42 milliwatts.

In addition, when the first medical instrument is a probe device, the first wavelength may be between 400 nanometers and 980 nanometers. In an embodiment, the first wavelength may be between 655 nanometers and 980 nanometers, the first frequency may be a steady stream or a frequency between 0 hertz and 5,000 hertz, and the first region may be approximately 0.28 centimeters squared in area in spot size or 6 millimeters in diameter. In addition, when the first medical instrument is a probe device, a treatment time required may be approximately 3 or more minutes on the biological medium, an energy produced through the first medical instrument may be approximately 17.5 millijoules per second, and a power generated may be less than 55 milliwatts.

Similarly, when the second medical instrument is a probe device, the second wavelength may be between 400 nanometers and 980 nanometers. In an embodiment, the second wavelength may be between 655 nanometers and 980 nanometers, the second frequency may be a steady stream or a frequency between 0 hertz and 5,000 hertz, and the second region may have an approximate diameter of 0.28 centimeters squared in area in spot size or 6 millimeters in diameter. In addition, when the second medical instrument is a probe device, a treatment time may be approximately 3 or more minutes on the biological medium, an energy produced through the second medical instrument may be approximately 17.5 millijoules per second, and a power generated may be less than 55 milliwatts.

The first soliton wave and the second soliton wave may be self-reinforcing solitary waves that maintain shape while traveling at a constant speed. The method may include applying the first soliton wave and the second soliton wave at different locations of a biological medium based on a requirement of a medical procedure. In addition, the method may include providing coordinated modes of operation between the first medical instrument and another medical instrument in a synchronous form, an asynchronous form, and a patterned form through a data processing system communicatively coupled to the first medical instrument and/or the second medical instrument. The first medical instrument and the second medical instrument may be a hand-held and portable medical instrument.

A battery of the first medical instrument and/or the second medical instrument may be a lithium-ion rechargeable battery that includes a power regulator to ensure stable and accurate delivery of power when generating the first soliton wave and/or the second soliton wave. In addition, the method may include operating the first medical instrument and the second medical instrument in a variety of operational modes. Each operational mode may be associated with a prescribed form of a medical treatment. The medical treatment may include an arthritic treatment, a diabetic treatment, a skeletal treatment, a muscle treatment, a musculoskeletal treatment, and/or a cardiatric treatment.

In addition, the method may include determining an appropriate operational mode based on an identification card in the first medical instrument and/or second medical instrument. The identification card may be removable by a user of the first medical instrument and second medical instrument. The laser and diode lights may be placed in a recessed upper portion of the first medical instrument and/or the second medical instrument.

In another aspect, a method includes coupling a plurality of laser-light based medical instruments to each other through a set of interfaces between laser-based medical instruments. The method also includes communicating a coordination command between a processor and the plurality of laser-based medical instruments. In addition, the method includes generating a soliton wave through the laser-based medical instruments responsive to the coordination command. In addition, the method may include coordinating a delivery of the soliton wave on a biological medium through control of delivery of laser and diode light of the plurality of laser-light based medical instruments.

In yet another aspect, a system includes a first medical instrument to produce a low-level laser light at a first frequency and at a first wavelength. In addition, the system includes a second medical instrument to produce a low-level laser light at a second frequency and at a second wavelength. The system also includes a processor to produce soliton waves through the first medical instrument and/or the second medical instrument when the first medical instrument and the second medical instrument are coupled to each other. The first and second frequencies may be a same frequency with the first and second wavelengths having a same wavelength.

The low-level laser light may be placed in a recessed upper portion of the first medical instrument and/or the second medical instrument. The low-level laser light in the recessed upper portion may be positioned in a form of a triangle equally spaced about a center of the recessed upper portion in a symmetrical manner. The system may also include a substantially planar diode array mounted in a recessed manner in the recessed portion of the first medical instrument and the second medical instrument. The substantially planar diode array may have a center and four sets of laser diodes each having a first, a second, and a third laser diode, and with each of the sets being arranged in an equilateral triangle. The sets of laser diodes may be equally spaced about a center of each of the sets, with the first laser diodes of each of the sets being spaced a first distance from a center of the diode array, the second and third laser diodes being spaced a greater second distance from the center of the diode array, and each of the first, second, and third laser diodes having a beam with the beams overlapping. The diode array may project a resultant composite beam that is directed at a biological medium to impart soliton energy on a biological medium.

The methods and systems disclosed herein may be implemented in any means for achieving various aspects, and may be executed in a form of a machine-readable medium embodying a set of instructions that, when executed by a machine, cause the machine to perform any of the operations disclosed herein. Other features will be apparent from the accompanying drawings and from the detailed description that follows.

### BRIEF DESCRIPTION OF THE DRAWINGS

The embodiments of this invention are illustrated by way of example and are not limited by the figures of the accompanying drawings, in which like references indicate similar elements and in which:

FIG. 1 is a system view that illustrates medical instruments being communicatively coupled and coordinated through a data processing system for treatment of a patient, according to one embodiment.

FIG. 2 is a schematic view of a primary device, according to one embodiment.

FIG. 3 is a schematic view of a probe device, according to one embodiment.

FIG. 4 is a detailed view illustrating the primary device of FIG. 2, according to one embodiment.

FIG. 5 is an alternative system view that illustrates the medical instruments being communicatively coupled and coordinated through a data processing system for treatment of the patient, according to another embodiment.

FIG. 6 is a schematic view illustrating a use of the medical instruments used for providing treatment to a biological medium, according to one embodiment.

FIG. 7 is a diagrammatic system view of the data processing system in which any of the embodiments disclosed herein may be performed, according to one embodiment.

FIG. 8A is a process flow illustrating generation and coordination of soliton waves through the medical instrument, according to one embodiment.

FIG. 8B is a continuation of a process flow of FIG. 8A, illustrating additional operations, according to one embodiment.

FIG. 9 is a process flow illustrating a process of generating and coordinating a delivery of the soliton wave on a biological medium, according to one embodiment.

FIG. 10 is a schematic view of a medical instrument, according to one or more embodiments.

FIG. 11 is a system view of a probe device, according to one or more embodiments.

FIG. 12 is a system view illustrating a mode server communicating an information associated with a mode to a medical instrument(s) through a client device(s) via a network, according to one or more embodiments.

Other features of the present embodiments will be apparent from the accompanying drawings and from the detailed description that follows.

### DETAILED DESCRIPTION

A method, an apparatus, and a system of a coordinated delivery of soliton waves through coupled medical instruments are disclosed. Although the present embodiments have been described with reference to specific example embodiments, it will be evident that various modifications and changes may be made to these embodiments without departing from the broader spirit and scope of the various embodiments.

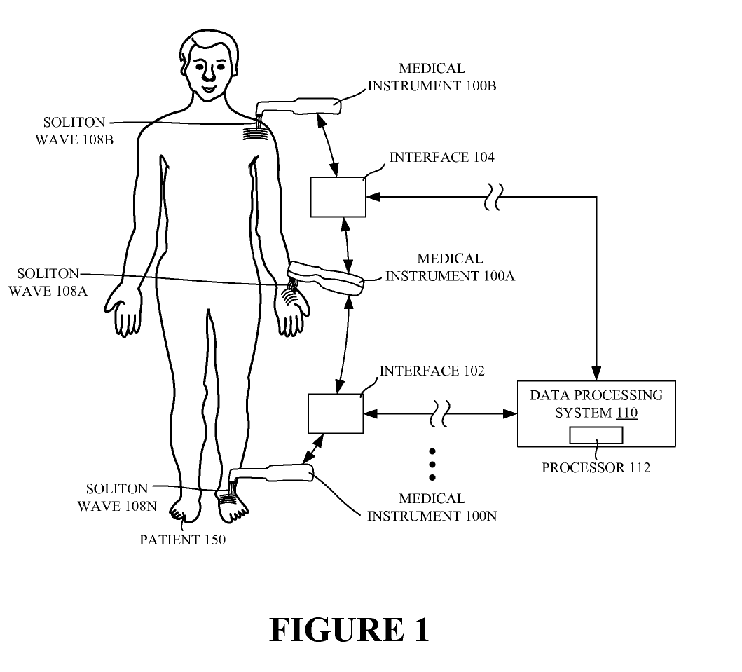

FIG. 1 is a system view that illustrates medical instruments 100A-N being communicatively coupled and coordinated through a data processing system 110 for treatment of a patient 150, according to one embodiment. In particular, FIG. 1 illustrates the medical instruments 100A-N, interfaces 102-104, soliton waves 108A-N, the data processing system 110, a processor 112, and the patient 150, according to one embodiment.

In one or more embodiments, soliton waves 108A-N may be generated from the one or more substantially planar laser diode(s). In one or more embodiments, end mirrors of the one or more substantially planar laser diode(s) may be replaced with anti-reflection coatings, and when the one or more substantially planar laser diode(s) are driven, the optical field evolution in the laser diode(s) may be modeled by using two coupled differential equations (example Equations 1 and 2) as:

$$\begin{matrix}\frac{\partial ψ}{\partial z} = \frac{i}{2} \frac{{\partial }^{2} ψ}{\partial {x}^{2}} + ( - i hN + ( N - 1 ) - α ) ψ , \\ and & ( 1 ) \\ D \frac{{\partial }^{2} N}{\partial {x}^{2}} = - π + N + {BN}^{2} + {CN}^{2} + ( N - 1 ) {\vert ψ \vert}^{2} , & ( 2 )\end{matrix}$$

where ψ may be the optical field solution, i=\sqrt{−1}, x and z the spatial coordinates, h the Henry factor, α the internal loss, N the normalized carrier density

$$( N = \frac{{N}^{′}}{{N}_{tr}^{′} , {N}^{′}}$$

being the carrier density, and N′ₜᵣ being the transparency carrier density), D the carrier diffusion coefficient, π the current pumping coefficient, B the spontaneous recombination coefficient, and C the Auger recombination rate. Here, a linear dependence of the induced refractive index and gain on the carrier density N may be assumed.

In one or more embodiments, neglecting carrier diffusion in the z direction, and assuming small diffusion, B=0, and C=0, a generalized complex Ginzburg-Landau equation may be obtained from Equations 1 and 2 as example Equation 3:

$$\frac{\partial ψ}{\partial z} = i ( \frac{1}{2} - i β ) \frac{{\partial }^{2} ψ}{\partial {x}^{2}} + ( \frac{π - 1}{1 + {\vert ψ \vert}^{2}} ( - i h + 1 ) - i h ) ψ - αψ , \qquad ( 3 )$$

where β may account for the transverse carrier diffusion.

In one or more embodiments, soliton wave solutions of the form ψ(x)e^{iλE} may be numerically obtained. In one or more embodiments, depending on the arrangement of the number of substantially planar laser diodes, constructive interference of the outputs of the number of substantially planar laser diodes may lead to a resultant soliton wave of high amplitude. In one or more embodiments, the resultant soliton wave output may have an amplitude several times higher than a non-soliton wave resultant beam.

In one embodiment, soliton waves 108A-N may be generated based on the positioning of the laser diodes. The pattern of the positioning of the laser diodes may affect the production and characteristics of soliton waves 108A-N.

The soliton wave 108A-N is a self-reinforcing solitary wave that maintains its shape while traveling at a constant speed. In an embodiment, soliton waves 108A-N may be generated using the medical instruments 100A-N. The medical instruments 100A-N illustrated are portable and hand-held devices. The soliton waves 108A-N may be generated by laser diodes embedded in the medical instruments 100A-N individually or in combination. The generation of soliton waves 108A-N in combination and coordination may be initiated by using an algorithm that controls the delivery of the soliton wave in the data processing system 110 that coordinates and controls a delivery of laser and diode light of the medical instruments 100A-N. The algorithm may be generated based on the requirement of a medical procedure.

The medical instruments 100A-N may be communicatively coupled to each other and to the data processing system 110 through the interfaces. The interfaces 102-104 may serve as a communication link between the medical instruments 100A-N. The data processing system 110 may be a computing device (e.g., computer) that includes the processor 112 to control the medical instruments 100A-N. The algorithm may be executed by the processor 112 in the data processing system 110 to initiate generation of soliton waves 108A-N in the medical instruments 100A-N in coordination. The algorithm that controls the delivery of the soliton waves may issue a coordination command to the processor 112 and the medical instruments 100A-N to generate a coordinated soliton waves 108A-N.

In the example embodiment, the soliton waves 108A-N are generated by the medical instruments 100A-N individually or in coordination by using the algorithm to coordinate the delivery of the soliton waves 108A-N. Each of the medical instruments 100A-N may be configured to generate a soliton wave at a particular frequency. In addition, the medical instruments 100A-N may be configured to operate in a particular mode. There may be a variety of operational modes for operating the medical instruments 100A-N in coordination. In one or more embodiments, the operational modes may be based on a prescribed form of a medical treatment. The data processing system 110 may coordinate and control the medical instruments 100A-N synchronously, asynchronously, or in a pattern. The soliton waves 108A-N generated, may be delivered on the biological mediums (e.g., such as a part of a body of the patient 150 that requires treatment) based on a medical procedure for various medical treatments. The medical treatments may include, but are not limited to, an arthritic treatment, a diabetic treatment, a skeletal treatment, a muscle treatment, a musculoskeletal treatment, and/or a cardiatric treatment.

In an example embodiment, the soliton waves 108A-N may be generated by canceling a nonlinear effect and a dispersive effect in a region between an emitting region of the medical instrument 100A-N and the biological medium 602. The dispersive effect may be a dispersion relationship (e.g., variation of wave propagation with wavelength or frequency of a wave) between a frequency and a speed of the soliton wave 108A-N.

For example, a patient John Doe may be suffering from arthritis. The patient John Doe may require treatments at bone joints in the shoulder and knee. The soliton wave generated by the medical instruments 100A-N may be used for treating joints of the patient John Doe. The medical instruments 100A-N may be used on John Doe's affected joints individually or in coordination based a prescribed form of medical treatment to provide temporary relief of pain and stiffness caused by arthritis. As the medical instruments 100A-N are controlled and coordinated through the data processing system 110, the patient John Doe may use it without difficulty of controlling the medical instruments 100A-N manually and without thinking about simultaneous controls. The soliton waves generated from the medical instruments may be delivered on John Doe's joints providing him relief from pain.

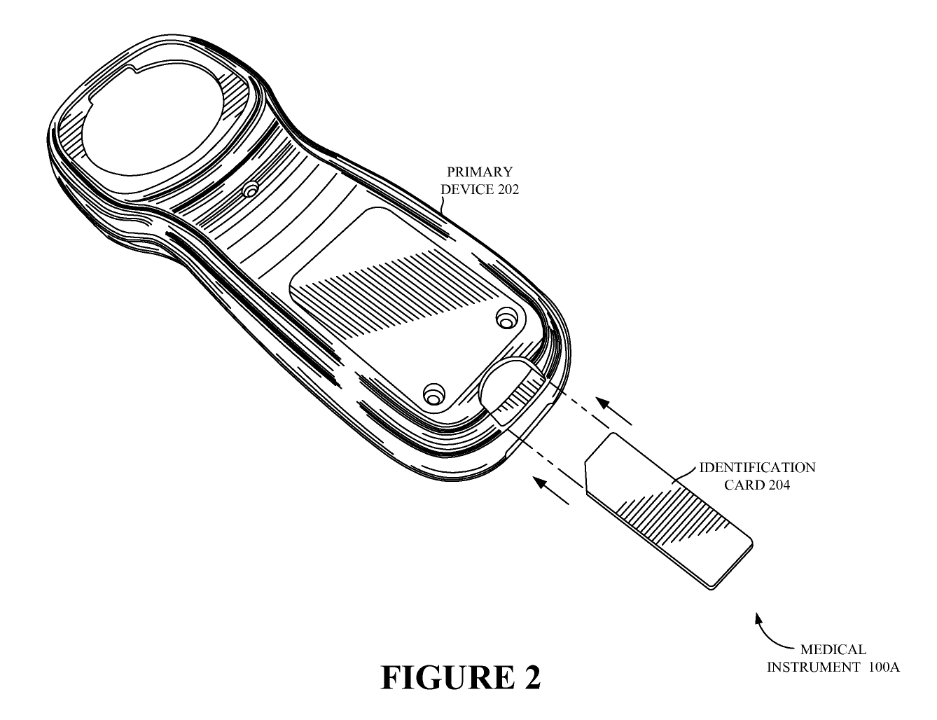

FIG. 2 is a schematic view of a primary device, according to one embodiment. In particular, FIG. 2 illustrates a primary device 202 and an identification card 204, according to one embodiment.

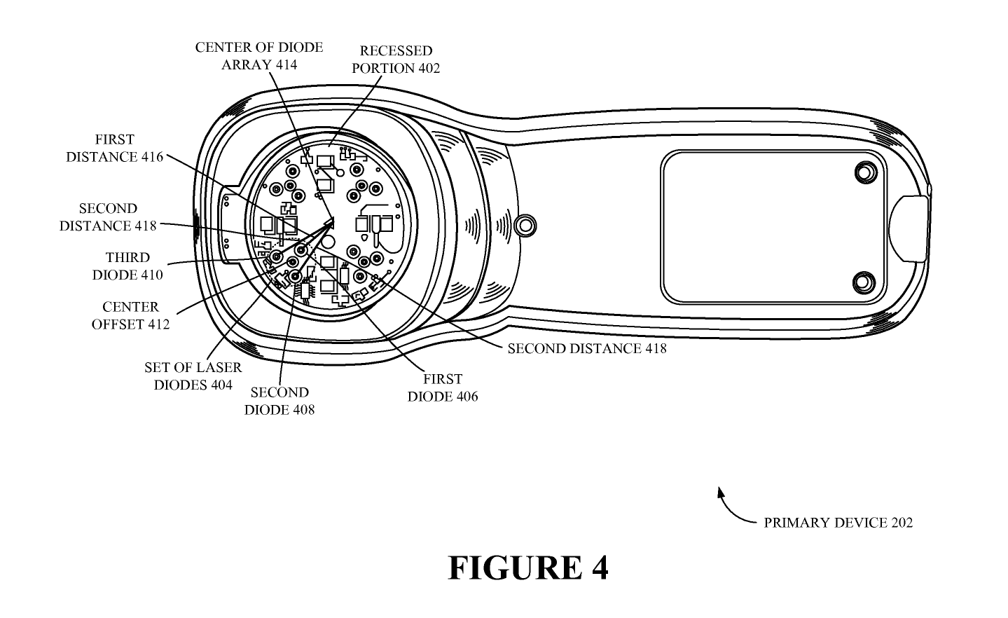

The medical instruments 100A-N illustrated in FIG. 1 may include primary devices and probe devices. The primary devices and the probe devices are portable and handheld devices. In an example embodiment, the medical instrument 100A may be the primary device 202. The laser diodes in the primary device 202 may be placed in a recessed portion 402 of the medical instrument 100A (e.g., as shown in FIG. 4). The primary device 202 may have a larger recessed portion 402 for radiating soliton waves as compared to a probe device 300. The primary device 202 may generate a wavelength between 645 nanometers and 811 nanometers and a frequency between 1 hertz and 20,000 hertz. In addition, a region may be approximately 16.4 centimeters squared in area in spot size, when the medical instrument 100A-N is the primary device 202. The treatment time required to treat the patient 150 when the medical instrument 100A-N is being used as the primary device 202 may be approximately 3 or more minutes. Furthermore, energy produced through the primary device 202 may be approximately 21 millijoules per second, and a power generated by the primary device 202 may be less than 42 milliwatts.

The primary device 202 described herein may include the identification card 204. The identification card 204 of the medical instrument 100A enables the medical instrument to operate in a certain operational mode. The identification card 204 coupled to the primary device 202 may be removable by a user of the medical instrument 100A. The operational modes may be associated with a prescribed form of a medical treatment. There may be variety of operational modes for a treating of a particular ailment. A doctor specialized and skilled in a treatment of a particular ailment may prescribe a best mode of treatment based on a medical condition of the patient 150.

In another embodiment, the patient 150 may obtain a diagnosis of a particular ailment from a medical professional. The medical instruments 100A-N may be available to patients pertaining to a particular ailment. In another embodiment, the medical instruments 100A-N may be available in stores.

Operational modes may be programmed into the primary device 202. The identification card 204 may activate the operational mode programmed into the primary device 202. Furthermore, the identification card 204 may be labeled based on a prescription associated to the condition of the patient 150, including for example, osteoarthritis or diabetes. The identification card 204 may be coupled to the primary device 202 through a port designated for the purpose. The primary device 202 may then generate soliton waves 108 based on the mode that is activated from the identification card 204.

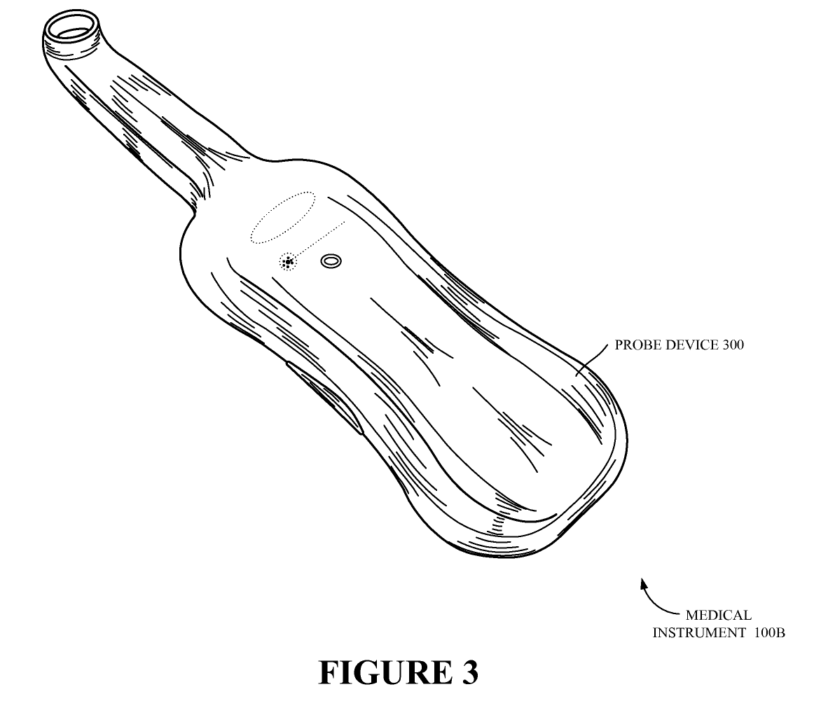

FIG. 3 is a schematic view of a probe device 300, according to one embodiment. The probe device 300 may have a small area for providing radiation for treatment unlike the primary device 202 which has a larger area providing radiation. The radiation as described may refer to the soliton waves generated in the medical instruments 100A-N. The probe device 300 may structurally include a port that may be used for coupling with either of the primary device 202, the interface 102-108 or the data processing system 110. In addition, the probe device 300 may include a button that enables the user of the probe device 300 to generate the radiation upon the button being pressed. In one embodiment, the probe device 300 may be used individually, in combination with the primary device 202, and/or in cluster with more than one medical instrument 100A-N. In one or more embodiments, the probe device 300 may also include an identification card for activating modes. The identification card may be communicatively coupled to the probe device 300 through a port provided thereof.

In an embodiment, the probe device 300 may generate a wavelength between 655 nanometers and 660 nanometers, a frequency that may be between 0 hertz and 5,000 hertz, and the region may be approximately 0.28 centimeters squared in area in spot size. Furthermore, a treatment time (e.g., radiation application time) required on the biological medium may be approximately 3 or more minutes using the probe device 300. In addition, an energy produced through the probe device 300 may be approximately 17.5 millijoules per second, with a power generated that may be less than 55 milliwatts.

FIG. 4 is a detailed view illustrating the primary device 202 of FIG. 2, according to one embodiment. Particularly, FIG. 4 illustrates a recessed portion 402, a set of laser diodes 404, a first diode 406, a second diode 408, a third diode 410, a center offset 412, a center of diode array 414, a first distance 416, and a second distance 418, according to one embodiment.

The primary device 202 may structurally include the recessed portion 402 that comprises circuitry to produce soliton waves 108. The laser diodes 404 may be placed in the recessed portion 402 of the primary device 202. The circuitry includes a substantially planar diode array mounted in a recessed manner in the recessed portion 402. The laser diode may be a semiconductor device that generates coherent radiation when powered. There may be, but not limited to, four sets of laser diodes in the recessed portion 402 that are carefully placed to produce the soliton waves 108. The set of laser diodes 404 may be placed at an equidistance from a point of the center of diode array 414. The set of laser diodes 404 may include the first diode 406, the second diode 408, and the third diode 410. The laser diodes in each of the set may be arranged in form of an equilateral triangle.

The first diode 406 of the set of laser diodes 404 may be placed at the first distance 416 from the center of the diode array 414. The second diode 408 and the third diode 410 may be placed at the second distance 418 from the center of the diode array 414. The second distance 418 may be greater than the first distance 416 from the center of the diode array 414. The center offset 412 may be the center of the set of laser diodes 404. Each of the set of laser diodes 404 may be spaced equidistant from the center of the diode array 414.

The same design may be implemented for the other set of laser diodes. The circuitry portion along with placement of the multiple set of laser diodes may be so designed to cancel a nonlinear effect and a dispersive effect in a region between an emitting region of the medical instrument 100A and the biological medium 602. Also, the placements of the diodes are such that a beam of the first diode 406, a beam of the second diode 408, and the beam of the third diode 410 overlap. Furthermore, a resultant composite beam generated from the diode array may be directed at the biological medium. The resultant composite beam may be used to impart energy on the biological medium such as a part of human body that requires treatment.

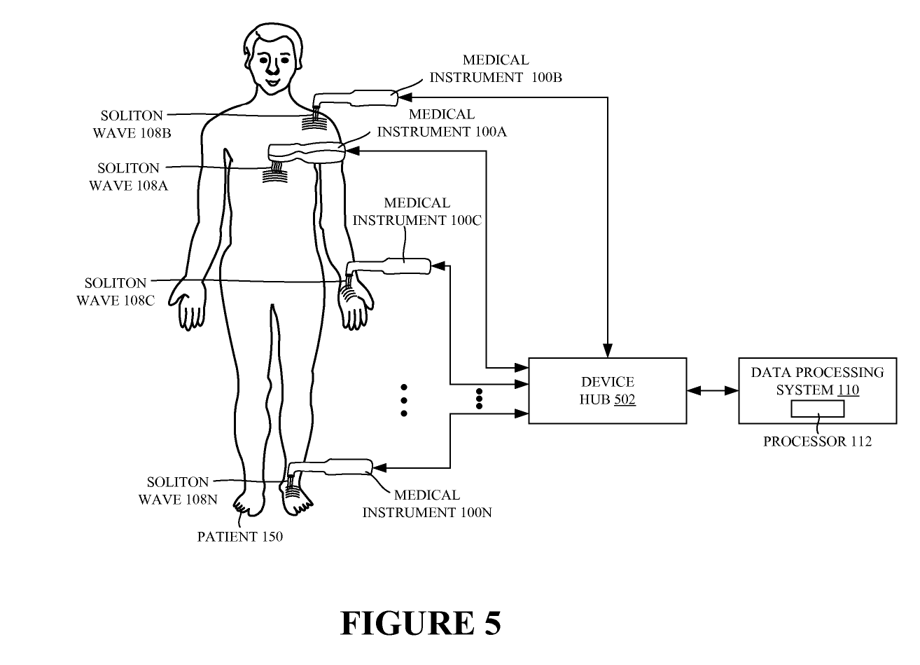

FIG. 5 is an alternative system view that illustrates the medical instruments being communicatively coupled and coordinated through a data processing system for treatment of the patient, according to another embodiment. In particular, FIG. 5 illustrates the patient 150, medical instruments 100A-N, a device hub 502, a data processing system 110, and a processor 112, according to one embodiment.

FIG. 5 provides an alternative embodiment to the system illustrated in FIG. 1. In an embodiment, the medical instruments 100A-N may be communicatively coupled to the data processing system 110 through the device hub 502. The device hub 502 may be a device that is used to connect the medical instruments 100A-N to the data processing system 110. In an example embodiment, the device hub 502 may serve as a bridge between the medical instruments 100A-N and the data processing system 110. Each of the medical instrument 100A-N may be connected to the device hub 502. The processor 112 in the data processing system 110 may control the generation and coordination of the soliton waves from the medical instruments 100A-N through the device hub 502.

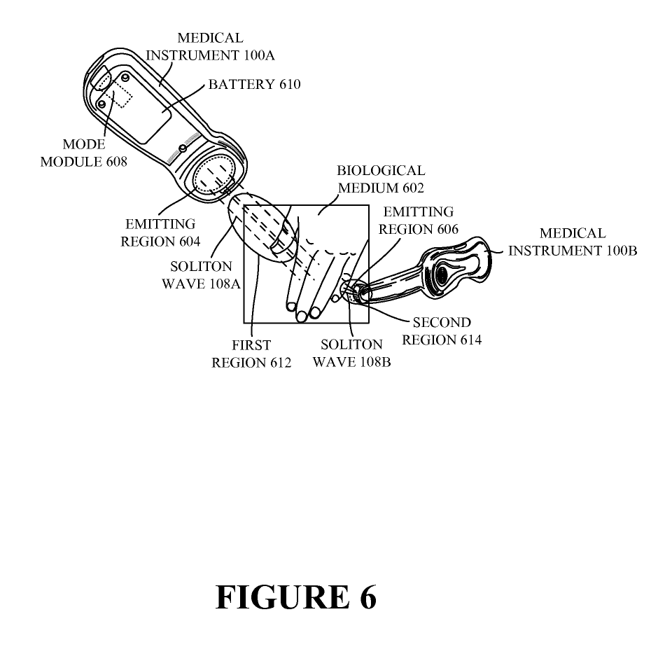

FIG. 6 is a schematic view illustrating a use of the medical instruments used for providing treatment to a biological medium, according to one embodiment. In particular, FIG. 6 illustrates a biological medium 602, an emitting region 604, an emitting region 606, a mode module 608, a battery 610, a first region 612, and a second region 614, according to one embodiment.

The biological medium 602 may be a part of a patient's body that has an ailment (e.g., related to arthritis). The medical instruments 100A-B may be used individually or in coordination. The emitting region 604 of the medical instrument 100A may include multiple sets of laser diodes carefully placed and supported by the associated circuitry to generate soliton waves. Similarly, the emitting region 606 of the medical instrument 100B may include a laser diode to generate a soliton wave 108. The medical instruments 100A-B may be powered using the battery 610. In an example embodiment, the battery 610 may be a lithium-ion rechargeable battery to power the medical instrument 100A-B. A power regulator of the battery 610 (not shown in the figure) may be used to provide stable and accurate power output to the medical instruments 100A-B. The mode module 608 may include a machine readable set of instructions to enable a mode. The mode module 608 may enable the circuitry to generate soliton waves based on a particular operational mode that is activated in the medical instruments 100A-B by the identification card 204. In one or more embodiments, the operational modes in the medical instruments 100A-B may also be programmed through a network (e.g., internet). The first region 612 may be the area of radiation being emitted from the medical instrument 100A. The second region 614 may be the area of radiation being emitted from the medical instrument 100B.

In an example embodiment, the soliton wave 108A may be generated by canceling a nonlinear effect and a dispersive effect in a first region 612 between an emitting region of the medical instrument 100A and the biological medium. In an example embodiment, the soliton wave 108B may be generated by canceling a nonlinear effect and a dispersive effect in a second region 614 between an emitting region of the medical instrument 100B and the biological medium. The dispersive effect may be a dispersion relationship (e.g., variation of wave propagation with wavelength or frequency of a wave) between a frequency and a speed of the soliton wave.

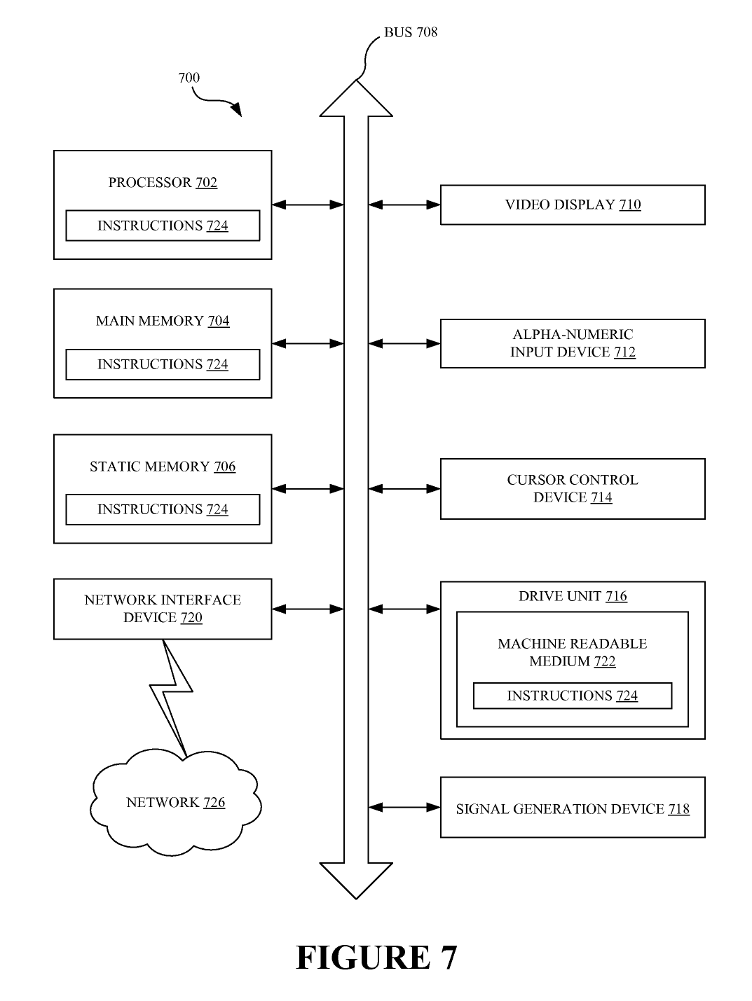

FIG. 7 is a diagrammatic system view 700 of the data processing system 110 in which any of the embodiments disclosed herein may be performed, according to one embodiment. Particularly, the diagrammatic system view 700 of FIG. 7 illustrates a processor 702, a main memory 704, a static memory 706, a bus 708, a video display 710, an alpha-numeric input device 712, a cursor control device 714, a drive unit 716, a signal generation device 718, a network interface device 720, a machine readable medium 722, instructions 724, and a network 726, according to one embodiment.

The diagrammatic system view 700 may indicate a personal computer and/or the data processing system 110 in which one or more operations disclosed herein are performed. The processor 702 may be a microprocessor, a state machine, an application specific integrated circuit, a field programmable gate array, etc. The main memory 704 may be a dynamic random access memory and/or a primary memory of a computer system.

The static memory 706 may be a hard drive, a flash drive, and/or other memory information associated with the data processing system 110. The bus 708 may be an interconnection between various circuits and/or structures of the data processing system 110. The video display 710 may provide graphical representation of information on the data processing system 110. The alpha-numeric input device 712 may be a keypad, a keyboard, and/or any other input device of text (e.g., a special device to aid the physically handicapped).

The cursor control device 714 may be a pointing device such as a mouse. The drive unit 716 may be the hard drive, a storage system, and/or other longer term storage subsystem. The signal generation device 718 may be a bios and/or a functional operating system of the data processing system 110. The network interface device 720 may be a device that performs interface functions such as code conversion, protocol conversion, and/or buffering required for communication to and from the network 726. The machine readable medium 722 may provide instructions 724 on which any of the methods disclosed herein may be performed. The instructions 724 may provide source code and/or data code to the processor 702 to enable any one or more operations disclosed herein.

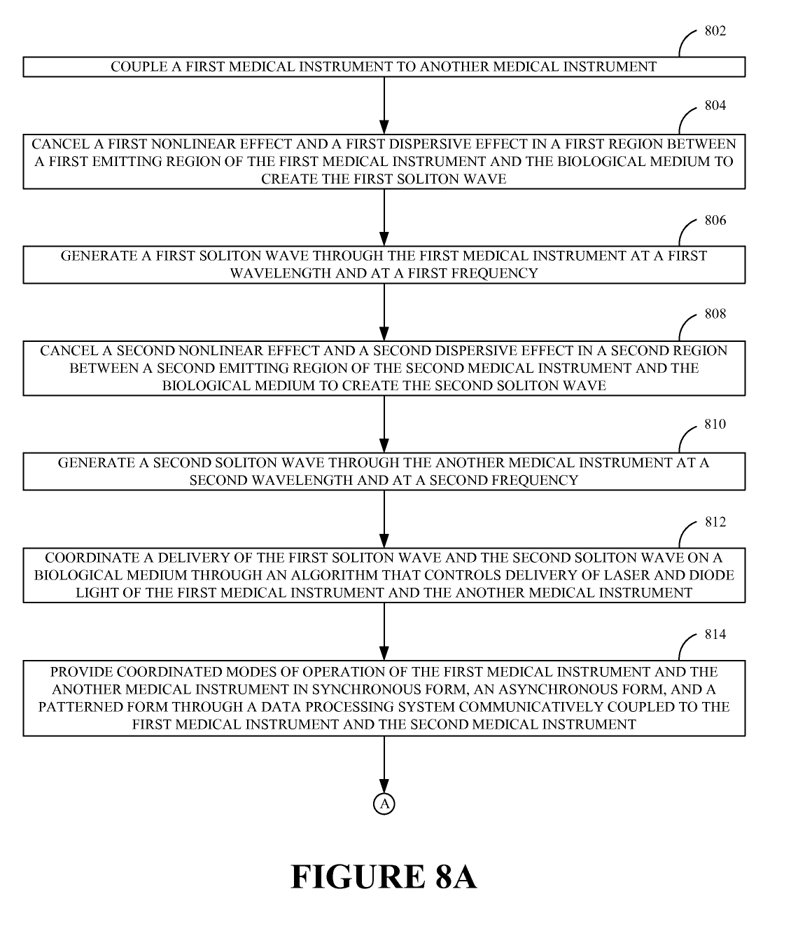

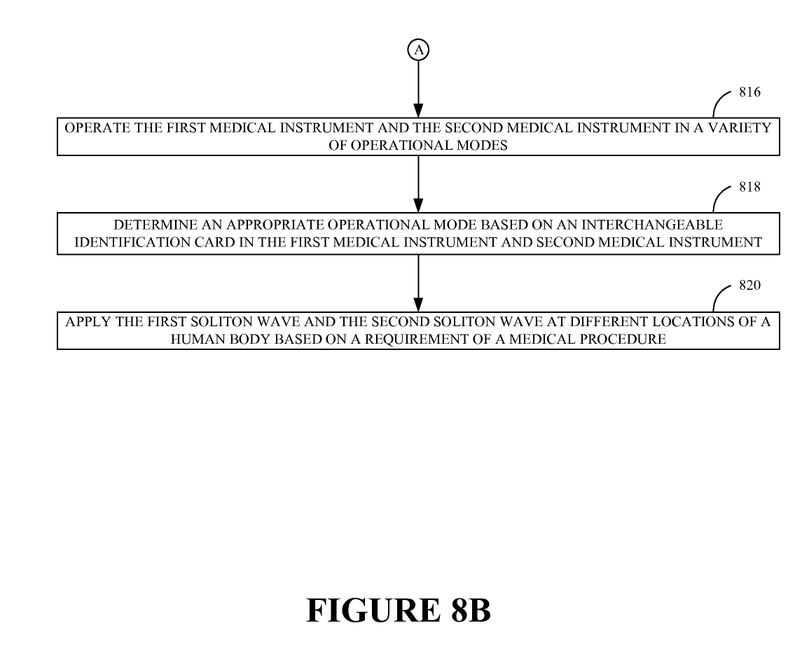

FIG. 8A is a process flow illustrating generation and coordination of soliton waves through the medical instrument, according to one embodiment. In operation 802, the first medical instrument 100A may be coupled with another medical instrument 100B (e.g., the second medical instrument). In operation 804, a first nonlinear effect and a first dispersive effect in a first region between the first emitting region 604 of the first medical instrument 100A and the biological medium 602 may be canceled to create the first soliton wave. In operation 806, the first soliton wave may be generated through the first medical instrument 100A at a first wavelength and at a first frequency. In operation 808, a second nonlinear effect and a second dispersive effect in a second region between the second emitting region 606 of the second medical instrument 100B and the biological medium 602 may be canceled to create the second soliton wave. In operation 810, the second soliton wave may be generated through another medical instrument 100B at a second wavelength and at a second frequency.

In operation 812, a delivery of the first soliton wave and the second soliton wave on the biological medium 602 may be coordinated through an algorithm that controls the start of a sequence of pulsation of the diodes from the medical instruments 100A-N affecting the delivery of laser and diode light of the first medical instrument 100A and another medical instrument 100B. In operation 814, coordinated modes of operation of the first medical instrument 100A and another medical instrument 100B may be provided in a synchronous form, an asynchronous form, or a patterned form through the data processing system 110 communicatively coupled to the first medical instrument 100A and the second medical instrument 100B.

FIG. 8B is a continuation of a process flow of FIG. 8A, illustrating additional operations, according to one embodiment. In operation 816, the first medical instrument 100A and the second medical instrument 100B may be operated in a variety of operational modes. In operation 818, an appropriate operational mode may be determined based on an identification card 204 in the first medical instrument 100A and/or the second medical instrument 100B. In operation 820, the first soliton wave and the second soliton wave may be applied at different locations of a human body based on a requirement of a medical procedure.

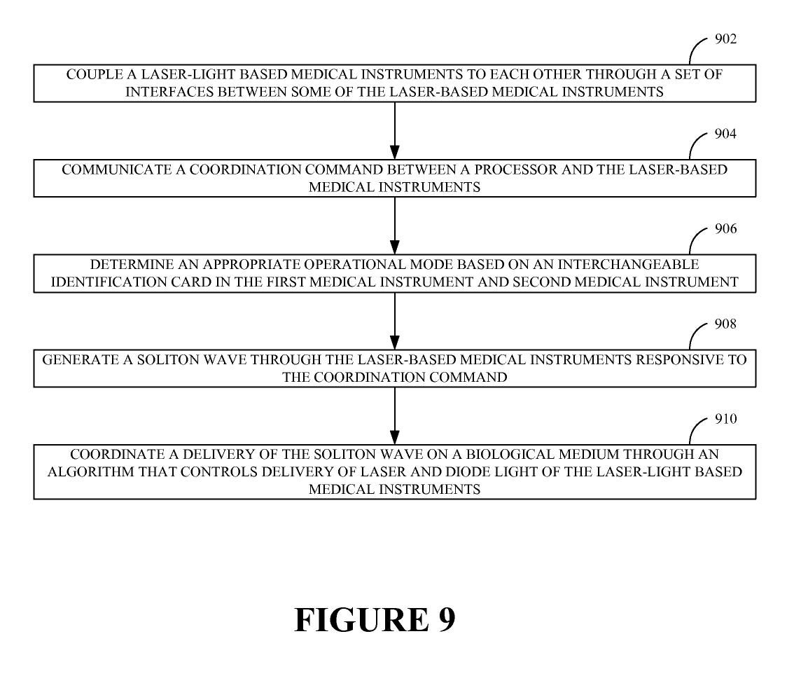

FIG. 9 is a process flow illustrating a process of generating and coordinating a delivery of soliton waves on a biological medium, according to one embodiment. In operation 902, laser-light based medical instruments (e.g., the medical instruments 100A-N) may be coupled to each other through a set of interfaces (e.g., the interfaces 102-104) between the laser-based medical instruments 100A-N. In operation 904, a coordination command may be communicated between the processor 112 and the laser-based medical instruments 100A-N. In operation 906, an appropriate operational mode may be determined based on the identification card 204 in the first laser-based medical instrument 100A and/or the second laser-based medical instrument 100B. In operation 908, the soliton waves may be generated through the laser-based medical instruments 100A-N responsive to the coordination command. In operation 910, a delivery of the soliton waves on a biological medium may be coordinated through an algorithm that controls the start of a sequence of pulsation of the diodes from the medical instruments 100A-N affecting the delivery of laser and diode light of the laser-light based medical instruments.

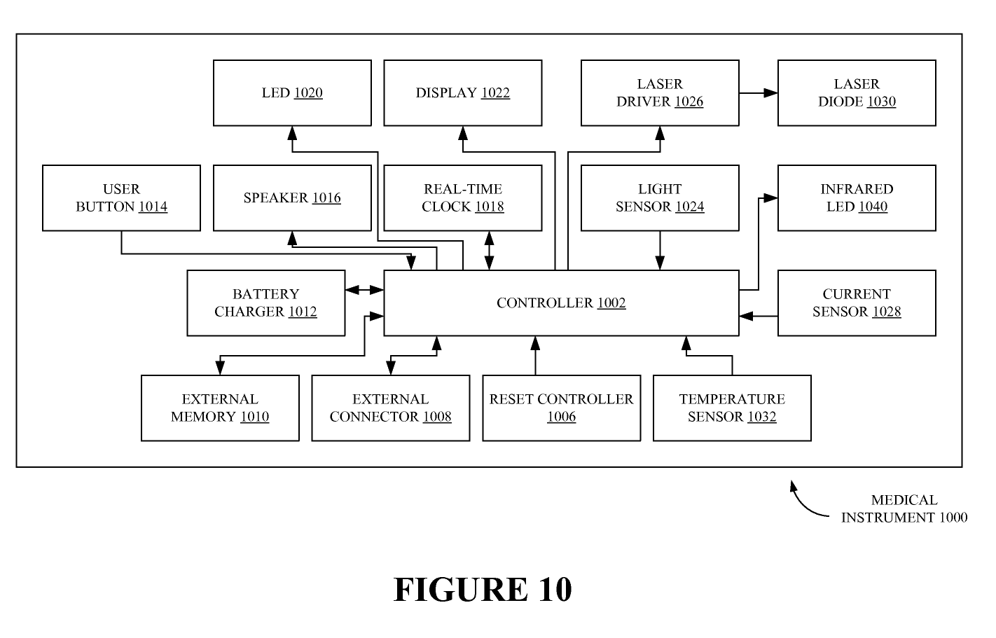

FIG. 10 is a schematic view of a medical instrument 1000, according to one or more embodiments. The medical instrument 1000 may in specific describe a schematic representation of the medical instrument 100A or the primary device 202. In one or more embodiments, the medical instrument 1000 may include a controller 1002 to control operations fundamental to the working of the medical instrument 1000. In one or more embodiments, the controller 1002 may include a permanent memory (e.g., flash memory) to store firmware associated with controlling the medical instrument 1000. In one or more embodiments, modes of operation may internally be set in the firmware. In one or more embodiments, the controller 1002 is interfaced with a battery charger 1012 to charge a battery (e.g., internal battery) of the medical instrument 1000. In one or more embodiments, the battery charging capability may be provided through an external connector 1008 that may serve purposes not limited to battery charging.

In one or more embodiments, the external connector 1008 may be a multi-pin and multi-use external connector that may also be used to program the internal controller of the medical instrument 1000 (e.g., controller 1002), to calibrate constituent laser diodes 1030, to couple other external compatible devices (e.g. another medical instrument 1000, a probe version of the medical instrument 1000, a computer device, a personal digital assistant (PDA)), and/or to perform diagnostics of the medical instrument 1000.

In one embodiment, the medical instrument 1000 may be powered by a lithium-ion rechargeable battery placed in an inside thereof. Here, the battery charger may plug into the medical instrument 1000 through the external connector 1008, and may closely monitor charge current as well as maximum allowed voltage. In one or more embodiments, the battery may be supplied with a safety circuitry to prevent over-charging/over-discharging of the battery. In one or more embodiments, constituent components of the medical instrument 1000 may be powered during charging of the battery, but user interaction with the medical instrument 1000 may not be possible.

In one or more embodiments, the controller 1002 may be interfaced with an external memory 1010 to enable the medical instrument 1000 to record data indicating a diagnostic requirement of the medical instrument 1000. In one or more embodiments, the recorded data may be useful in enabling servicing of the medical instrument 1000. For example, corrective diagnostics may be performed on the medical instrument 1000 by service personnel following a return of the medical instrument 1000 by a user. In one or more embodiments, the external memory 1010 may be a non-volatile memory such as an Electrically Erasable Programmable Read-Only Memory (EEPROM).

In one or more embodiments, the medical instrument 1000 may be provided with a user button 1014 (shown in FIG. 10 as turning on the controller 1002) to simplify operations thereof. In one embodiment, the user button 1014 may serve as both the power ON/OFF button and the mode selection button.

In one or more embodiments, the medical instrument 1000 may be provided with a speaker 1016 (shown in FIG. 10 as being controlled by the controller 1002) to generate audible alerts as well as indicate the pressing of the user button 1014. In one or more embodiments, the audible alerts may indicate one or more of an operational status of the medical instrument 1000, a beginning of a mode of operation, a beginning of a segment, an end of a mode of operation, and an end of the segment. In one or embodiments, all audible alerts may be muted by the user during use of the medical instrument 1000.

In one or more embodiments, to enhance serviceability of the medical instrument 1000, a real-time clock 1018 (shown in FIG. 10 as being interfaced with the controller 1002) may be implemented in the medical instrument 1000. In one or more embodiments, data recorded in the external memory 1010 may always be tagged with a current date and time at the time of recording. In one or more embodiments, this may enable a history of use of the medical instrument 1000 to be tracked. For example, when a medical instrument 1000 is returned to the service personnel, the service personnel may be better equipped to understand problems associated with the functioning of the medical instrument 1000.

In one or more embodiments, the medical instrument 1000 may be equipped with one or more Light Emitting Diodes 1020 (LEDs) and a display 1022 (e.g., seven segment display) that serve as user indicators. In FIG. 10, the LEDs 1020 and the display 1022 are shown as being controlled by the controller 1002. In one embodiment, the operational state of the medical instrument 1000 may be indicated with an LED emitting green light that may turn red during a power down. Here, another LED may be provided to indicate battery state and battery charging. For example, if the light emitted by this LED turns yellow during normal operation, it may be indicative of a low power level of the battery. The battery may then need to be charged. The LED may emit red light in a blinking state until charging may be complete, following which the LED may continue to emit green light. In one or more embodiments, the display 1022 may indicate modes that are loaded onto the medical instrument 1000, and, in one embodiment, the modes may be indicated on the display as 0-9. Here, the user may select a mode using the mode selection feature of the user button 1014.

In one or more embodiments, one of the purposes of the controller 1002 may be to control the laser diodes 1030 through laser drivers 1026 thereof. In one or more embodiments, the controller 1002 may control the power level of the laser diodes 1030, and also the flashing of the laser diodes 1030. In addition, in one or more embodiments, the controller 1002 may monitor a light sensor 1024 that measures the ambient light outside the medical instrument 1000. This measurement may be used to control the light intensity of the user indicator LEDs 1020.

In one or more embodiments, the controller 1002 may have the ability to sense the operating current of each laser diode 1030 (see current sensor 1028 in FIG. 10), which may be used to deactivate laser diodes 1030 that may have failed. In one or more embodiments, this may ensure safety of operation of the medical instrument 1000. In one or more embodiments, current may also be sensed during calibration of the medical instrument 1000 to ensure proper operation of the laser diodes 1030. In one or more embodiments, a power management circuitry of the laser diodes 1030 may be controlled by the controller 1002. In one or more embodiments, infrared light may also be emitted from the infrared LEDs 1040.

In one or more embodiments, the medical instrument 1000 may also include a number of infrared LEDs 1040 (shown as being controlled in FIG. 10 by the controller 1002) to emit infrared light during a duration of a mode of operation. In one or more embodiments, the infrared LEDs 1040 may operate in conjunction with one or more of the visible LEDs 1020.

In one or more embodiments, the controller 1002 may monitor a temperature sensor 1032 to obtain accurate values of the temperatures of the laser diodes 1030. In one or more embodiments, variations of temperature of the laser diodes 1030 may also be tracked.

In one or more embodiments, the medical instrument 1000 may include a reset controller 1006 to monitor a reset button. For example, when a user depresses the reset button and holds the reset button for, say, 5 seconds, the reset controller 1006 may send a reset signal to the controller 1002 to reset the medical instrument 1000. Here, 5 seconds is the threshold time period; and if a user presses the reset button for a time period exceeding the threshold time period, the medical instrument 1000 may be reset.

In one or more embodiments, when the medical instrument 1000 is turned ON and is in an idle state, an LED 1020 indicating power may emit green light. In one or more embodiments, a shut off timer may be started internally to turn the medical instrument 1000 off in case of inactivity (e.g., no further pressing of buttons) for a time period exceeding another threshold time period.

In one or more embodiments, the medical instrument 1000 may be pre-programmed (e.g., by the manufacturer) with several operational modes. In one or more embodiments, the modes may be pre-programmed with the duration of treatment for a therapeutic condition, and the specific frequencies the medical instrument 1000 may be operating at.

In one or more embodiments, where there is a requirement of directed, high-power dosage in a narrow region of a biological medium, the second medical instrument 100B, for example, may be a probe device.

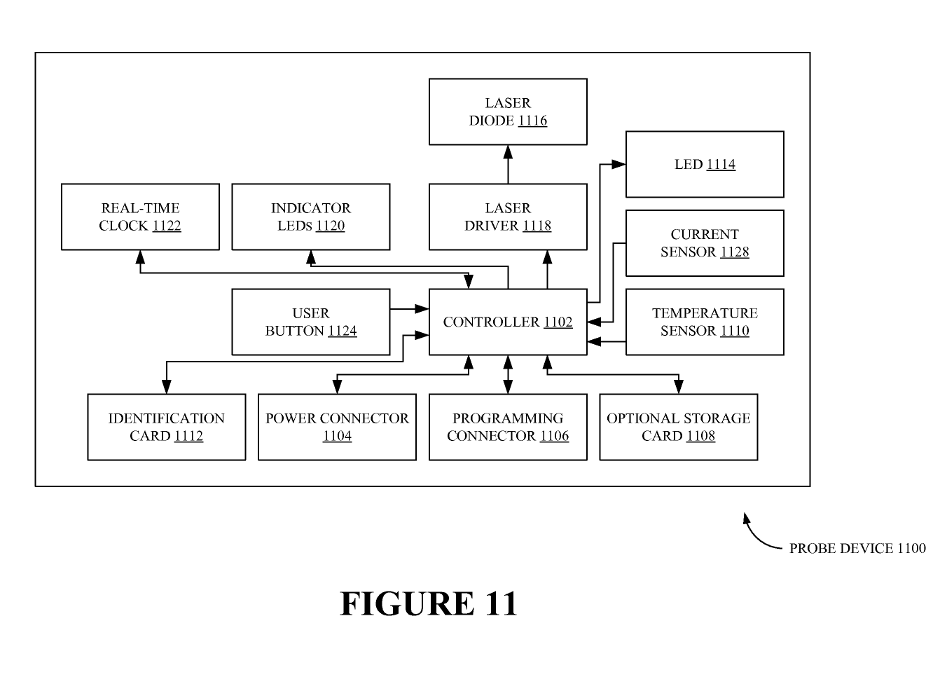

FIG. 11 is a system view of a probe device 1100, according to one or more embodiments. The probe device 1100 described herein may be substantially similar or the same as medical instrument 100B. In one or more embodiments, the prove device 1100 described herein may be a schematic representation of the medical instrument 100B. In one or more embodiments, the probe device 1100 may include a controller 1102 to control all components of the probe device 1100. In one or more embodiments, an operating program of the controller 1102 may be user-upgraded using an optional storage card 1108. In one or more embodiments, the optional storage card 1108 may be a flash card from which different programs may be read.

In one or more embodiments, the probe device 1100 may include a power connector 1104 through which a battery of the probe device 1100 may be charged. In one or more embodiments, a medical instrument 100B may be used to power the probe device 1100 through the power connector 1104. In one or more embodiments, the probe device 1100 may include an identification card 1112. The identification card 1112 may include information regarding types of treatment modes to be activated. The information on the identification card 1112 may be read by controller 1102.

In one or more embodiments, the probe device 1100 may include a programming connector 1106 through which a programming/calibration interface may be provided. In one of more embodiments, the probe device 1100 may be calibrated by a manufacturer and/or serviced by service personnel through the programming connector 1106. In one or more embodiments, a data processing system 110 may be coupled to the probe device 1100 through the programming connector 1106. In one or more embodiments, the programming connector 1106 may not be available to a user but only available to the manufacturer and/or service personnel.

In one or more embodiments, an integrated laser driver 1118 may control a laser diode 1116 of the probe device 1100. In one or more embodiments, an operating current of the laser diode 1116 and/or a light output of the laser diode 1116 may be monitored to maintain a constant output of the laser diode 1116. In one or more embodiments, the laser diode 1116 may be calibrated during the manufacturing process and/or the laser driver 1118 may be configured to handle a range of laser diodes.

In one or more embodiments, LEDs (1114, 1120) may be provided to indicate an operational state of the probe device 1100. A light from an LED 1114 may also indicate that the optional storage card 1108 is properly inserted and recognized. In another example, a number of LEDs 1120 may indicate modes selected and/or progress during boot-up. In one or more embodiments, a separate LED 1114 may indicate activity of the laser diode 1116.

In one or more embodiments, in order for corrective diagnostics to be performed by service personnel and/or operating statistics to be obtained by the manufacturer, a real-time clock 1122 may be provided in the probe device 1100. In one or more embodiments, the real-time clock 1122 may be programmed during manufacturing. In one embodiment, power to the real-time clock 1122 may be supplied by a lithium-ion battery of the probe device 1100. In another embodiment, power to the real-time clock 1122 may be supplied by a coin cell battery of the probe device 1100.

In one or more embodiments, the controller 1102 may monitor the current of the laser diode 1116 during operation of the laser diode 1116 through a current sensor 1128. In one embodiment, the current data may be used in the calibration of the probe device 1100.

In one or more embodiments, a temperature sensor 1110 may be provided in the probe device 1100 to monitor a temperature of the laser diode 1116 in order to ensure safety of operation of the probe device 1100.

In one or more embodiments, when the probe device 1100 is powered up, green light may be emitted from an LED 1120. In one embodiment, when the optional storage card 1108 is not present, the green LED 1120 may start to blink to indicate the need to insert the optional storage card 1108. In one or more embodiments, upon insertion of the identification card 1112 and checking for updates residing in the identification card 1112, modes of operation may be downloaded into the probe device 1100. In one or more embodiments, modes of operation present on the identification card 1110 may be loaded.

In one or more embodiments, user selection of modes of operation may be accomplished through a user button 1124. In one or more embodiments, the probe device 1100 may be turned on by a user holding the user button 1124 for a time period exceeding a threshold time period of, say, 5 seconds. In one or more embodiments, a warning LED 1114 may be provided to indicate a state where the laser diode 1116 operating at a wavelength outside the visible spectrum may be used. In one or more embodiments, the probe device 1100 may also be turned off by a user depressing the user button 1124 for a time period exceeding another threshold time period.

In one or more embodiments, if at any point the identification card 1112 is removed, the laser diode 1116 may be turned off, and the probe device 1100 may return to a boot-up state thereof.

In one or more embodiments, one or more substantially planar laser diode(s) of medical instrument 100B may lase at a wavelength of approximately ˜650 nm, ˜780 nm or ˜808 nm. In one or more embodiments, the medical instrument 100B may operate at a power level of approximately ˜60 mW. In one or more embodiments, the probe device 1100 may lase at a wavelength of approximately ˜660 nm or ˜808 nm. In one or more embodiments, the probe device 1100 may operate at a power level of approximately ˜50 mW or ˜500 mW. In one or more embodiments, the high power level of the probe device 1100 may provide for deeper penetration into a biological medium (e.g., tissue in a human body).

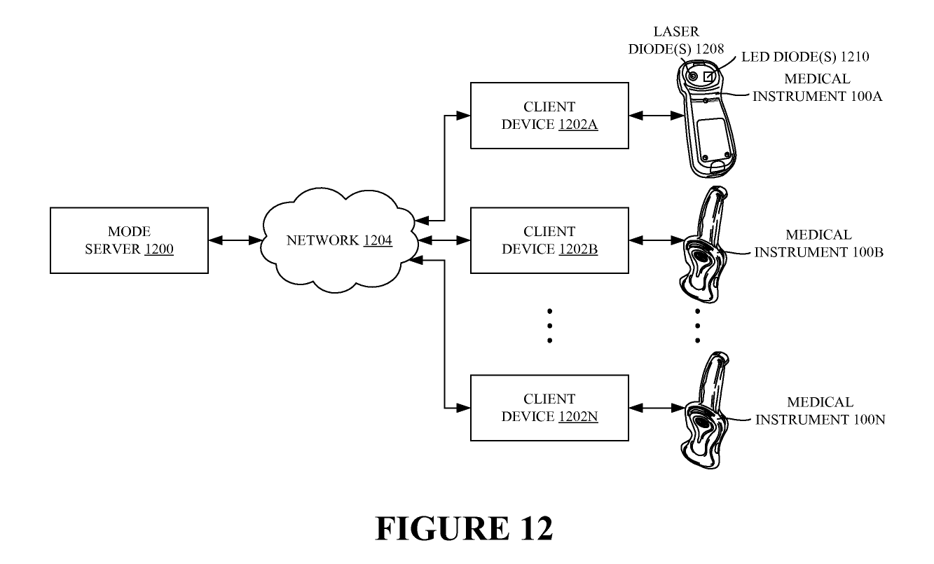

FIG. 12 is a system view illustrating a mode server 1200 communicating an information associated with a mode to a medical instrument(s) 100A-N through a client device(s) 1202A-N via a network 1204, according to one or more embodiments. Particularly, FIG. 12 illustrates the mode server 1200, the client device(s) 1202A-N, the network 1204, the medical instrument(s) 100A-N, a laser diode(s) 1208, and a LED(s) 1210, according to one or more embodiments. It should be noted that the medical instruments described herein the Figure are substantially similar or the same as illustrated in previous Figures. Also, the client device(s) 1202A-N described herein may be substantially similar or the same as illustrated in previous figures.

The mode server 1200 may provide different modes of operation for the medical instruments 100A-N via the network 1204. The client device 1202A-N may be any computing device (e.g., the data processing system 110) that can interface the medical instrument 100A-N for communicating the mode of operation to the medical instrument 100A-N. The mode may control the laser diodes 1208 and the LED diodes 1210 to generate a laser wavelength based on the mode. In one or more embodiments, the mode may configure the laser diodes 1208 and the LED diodes 1210 to generate laser at different wavelengths. In one or more embodiments, the client device 1202A-N may include, but is not limited to, a computer. In one or more embodiments, the client device 1202A-N upon receiving the information may provide an acknowledgment to the mode server via the network 1204. In one or more embodiments, the information associated with the mode may include, but is not limited to, a mode configuration, setting information, and handling instructions. In one or more embodiments, the mode server 1200 may be supported by a custom mode database (not shown in the Figure). The custom mode database may be a central resource for information associated with the modes. In one or more embodiments, a custom mode of operation may be configured into the medical instrument 100A-N and the treatment based on the custom mode may be provided to the user. In one or more embodiments, the custom mode configured by the user may be communicated to the mode server 1200 through the client device 1202A-N via the network 1204.

Although the present embodiments have been described with reference to specific example embodiments, it will be evident that various modifications and changes may be made to these embodiments without departing from the broader spirit and scope of the various embodiments. For example, the various devices and modules described herein may be enabled and operated using hardware circuitry, firmware, software, or any combination of hardware, firmware, and software (e.g., embodied in a machine readable medium 722). For example, the various electrical structure and methods may be embodied using transistors, logic gates, and electrical circuits (e.g., application specific integrated (ASIC) circuitry).

In addition, it will be appreciated that the various operations, processes, and methods disclosed herein may be embodied in a machine-readable medium and/or a machine accessible medium compatible with a data processing system (e.g., a computer system), and may be performed in any order (e.g., including using means for achieving the various operations). Accordingly, the specification and drawings are to be regarded in an illustrative manner rather than a restrictive sense.

What is claimed:

1. A method comprising:
coupling a first medical instrument to a second medical instrument;
generating a first soliton wave through the first medical instrument at a first wavelength and at a first frequency;
generating a second soliton wave through the second medical instrument at a second wavelength and at a second frequency; and
coordinating a delivery of the first soliton wave and the second soliton wave on a biological medium through an algorithm that controls a start of a sequence of pulsation of the diodes from the different instruments affecting the delivery of laser and diode light of the first medical instrument and the second medical instrument.

2. The method of claim 1 further comprising:
canceling a first nonlinear effect and a first dispersive effect in a first region between a first emitting region of the first medical instrument and the biological medium to create the first soliton wave, wherein the first dispersive effect is a first dispersion relationship between the first frequency and a first speed of the first soliton wave; and
canceling a second nonlinear effect and a second dispersive effect in a second region between a second emitting region of the second medical instrument and the biological medium to create the second soliton wave, wherein the second dispersive effect is a second dispersion relationship between the second frequency and a second speed of the second soliton wave.

3. The method of claim 2 wherein when at least one of the first medical instrument and the second medical instrument is a primary device,
the first and second wavelengths are between 645 nanometers and 811 nanometers,
the first and second frequencies are between 1 hertz and 20,000 hertz,
the first region and the second region are approximately 16.4 centimeters squared in area each in spot size,
a treatment time is approximately 3 minutes on the biological medium,
an energy produced through at least one of the first primary device and the second primary device is approximately 21 millijoules per second, and
a power generated is less than 42 milliwatts.

4. The method of claim 2 wherein when at least one of the first medical instrument and the second medical instrument is a probe device,
the first and second wavelengths are between 655 nanometers and 660 nanometers,
the first and second frequencies are between 0 hertz and 5,000 hertz,
the first region and the second region are approximately 0.28 centimeters squared in area each in spot size,
a treatment time is approximately 3 minutes on the biological medium,
an energy produced through at least one of the first probe device and the second probe device is approximately 17.5 millijoules per second, and
a power generated is less than 55 milliwatts.

5. The method of claim 1 wherein the first soliton wave and the second soliton wave are self-reinforcing solitary waves that maintain shape while traveling at a constant speed.

6. The method of claim 1 further comprising:
applying the first soliton wave and the second soliton wave at different locations of a human body based on a requirement of a medical procedure.

7. The method of claim 1 further comprising:
providing coordinated modes of operation of the first medical instrument and another medical instrument in at least a synchronous form, an asynchronous form, and a patterned form through a data processing system communicatively coupled to the first medical instrument and the second medical instrument,
wherein at least one of the first medical instrument and the second medical instrument is a hand-held and portable medical instrument.

8. The method of claim 1 wherein a battery of at least one of the first medical instrument and the second medical instrument is a lithium-ion rechargeable battery that includes a power regulator that ensures stable and accurate delivery of power when generating at least one of the first soliton wave and the second soliton wave.

9. The method of claim 1 further comprising:
operating the first medical instrument and the second medical instrument in a variety of operational modes, wherein each operational mode is associated with a prescribed form of a medical treatment,
wherein the medical treatment is at least one of an arthritic treatment, a diabetic treatment, a skeletal treatment, a muscle treatment, a musculoskeletal treatment, and a cardiatric treatment.

10. The method of claim 9 further comprising:
determining an appropriate operational mode based on an identification card in at least one of the first medical instrument and the second medical instrument, wherein the identification card is removable by a user of at least one of the first medical instrument and the second medical instrument.

11. The method of claim 10 wherein the laser and diode lights are placed in a recessed upper portion of at least one of the first medical instrument and the second medical instrument.

12. A method comprising:
coupling a plurality of laser-light based medical instruments to each other through a set of interfaces between at least some of the plurality of laser-based medical instruments;
communicating a coordination command between a processor and the plurality of laser-based medical instruments; and
generating a soliton wave through the laser-light based medical instruments responsive to the coordination command.

13. The method of claim 12 further comprising coordinating a delivery of the soliton wave on a biological medium through an algorithm that controls the start of a sequence of pulsation of the diodes from the different instruments affecting the delivery of laser and diode light of the plurality of laser-light based medical instruments.

14. The method of claim 12 wherein when at least one of the first medical instrument and the second medical instrument is a primary device,
the first and second wavelengths are between 645 nanometers and 811 nanometers,
the first and second frequencies are between 1 hertz and 20,000 hertz,
a first region and a second region are approximately 16.4 centimeters squared in area each in spot size,
a treatment time is approximately 3 minutes on the biological medium,
an energy produced through at least one of the first primary device and the second primary device is approximately 21 millijoules per second, and
a power generated is less than 42 milliwatts.

15. The method of claim 12 wherein when at least one of the first medical instrument and the second medical instrument is a probe device,
the first and second wavelengths are between 655 nanometers and 660 nanometers,
the first and second frequencies are between 0 hertz and 5,000 hertz,
the first region and the second region are approximately 0.28 centimeter diameter each in spot size,
a treatment time is approximately 3 minutes on the biological medium,
an energy produced through at least one of the first probe device and the second probe device is approximately 17.5 millijoules per second, and
a power generated is less than 55 milliwatts.

16. The method of claim 12 further comprising:
determining an appropriate operational mode based on an identification card in at least one of the first medical instrument and the second medical instrument, wherein the identification card is removable by a user of at least one of the first medical instrument and the second medical instrument.

17. The method of claim 12 wherein the laser and diode lights are placed in a recessed upper portion of at least one of the first medical instrument and the second medical instrument.

18. A system comprising:
a first medical instrument to produce a low-level laser light at a first frequency and at a first wavelength;
a second medical instrument to produce a low-level laser light at a second frequency and at a second wavelength; and
a processor to produce soliton waves through at least one of the first medical instrument and the second medical instrument when the first medical instrument and the second medical instrument are coupled to each other.

19. The system of claim 18 wherein the first and second frequencies are a same frequency and wherein the first and second wavelengths are a same wavelength.

20. The system of claim 18 wherein when at least one of the first medical instrument and the second medical instrument is a primary device,
the first and second wavelengths are between 645 nanometers and 811 nanometers,
the first and second frequencies are between 1 hertz and 20,000 hertz,
a first region and a second region are approximately 16.4 centimeter diameter each in spot size,
a treatment time is approximately 3 minutes on a biological medium,
an energy produced through at least one of the first primary device and the second primary device is approximately 21 millijoules per second, and
a power generated is less than 42 milliwatts.

21. The system of claim 18 wherein when at least one of the first medical instrument and the second medical instrument is a probe device,
the first and second wavelengths are between 655 nanometers and 660 nanometers,
the first and second frequencies are between 0 hertz and 5,000 hertz,
the first region and the second region are approximately 0.28 centimeters squared in area each in spot size,
a treatment time is approximately 3 minutes on the biological medium,
an energy produced through at least one of the first probe device and the second probe device is approximately 17.5 millijoules per second, and
a power generated is less than 55 milliwatts.

22. The system of claim 18 further comprising:
an identification card in at least one of the first medical instrument and the second medical instrument to set an appropriate operational mode, wherein the identification card is removable by a user of at least one of the first medical instrument and the second medical instrument.

23. The system of claim 18 wherein the low-level laser light is placed in a recessed upper portion of at least one of the first medical instrument and the second medical instrument.

24. The system of claim 23 wherein the low-level laser light in the recessed upper portion is positioned in a form of a triangle equally spaced about a center of the recessed upper portion in a symmetrical manner.

25. The system of claim 18 further comprising:
a substantially planar diode array mounted in a recessed manner in a recessed portion of at least one of the first medical instrument and the second medical instrument, and having a center and at least four sets of laser diodes each having a first, a second, and a third laser diode, with each of the sets being arranged in an equilateral triangle, the sets being equally spaced about the center of each of the sets with the first laser diodes of each of the sets being spaced a first distance from a center of the diode array, and the second and third laser diodes being spaced a greater second distance from the center of the diode array, and each of the first, second, and third laser diodes having a beam, the beams overlapping, and
whereby the diode array projects a resultants composite beam that is directed at the biological medium to impart soliton energy on a biological medium.
